(19) United States
(12) Patent Application Publication
(10) Pub. No.: US 2006/0024676 A1
(43) Pub. Date: Feb. 2, 2006

(54) Method of detecting epigenetic biomarkers by quantitative methyISNP analysis

(76) Inventors: Karen Uhlmann, Berlin (DE); Peter Nurnberg, Berlin (DE); Anja Brinckmann, Berlin (DE)

Correspondence Address: JOYCE VON NATZMER, 4615 NORTH PARK AVENUE, SUITE 919, CHEVY CHASE, MD, 20815, US

(21) Appl. No.: 10/823,784

(22) Filed: Apr. 14, 2004

Related U.S. Application Data
(60) Provisional application No. 60/462,289, filed on Apr. 14, 2003.

(51) Int. Cl. C12Q 1/68

(52) U.S. Cl. 435/6

(57) ABSTRACT

The present invention relates to a method for the detection of the methylation status of a nucleotide at a predetermined position in a nucleic acid molecule comprising the steps of (a) treating a sample comprising said nucleic acid molecule or consisting of said nucleic acid molecule in an aqueous solution with an agent suitable for the conversion of said nucleotide if present in (i) methylated form; or (ii) non-methylated form to pair with a nucleotide normally not pairing with said nucleotide prior to conversion; (b) amplifying said nucleic acid molecule treated with said agent; (c) real-time sequencing said amplified nucleic acid molecule; and (d) detecting whether said nucleotide is formerly methylated or not methylated in said predetermined position in the sample. The invention further relates to a method for the diagnosis of a pathological condition or the predisposition for a pathological condition comprising detection of a methylation status nucleotide at a predetermined position in a nucleic acid molecule comprising the steps of (a) treating a sample comprising said nucleic acid molecule or consisting of said nucleic acid molecule in an aqueous solution with an agent suitable for the conversion of said nucleotide if present in (i) methylated form; or (ii) non-methylated form to pair with a nucleotide normally not pairing with said nucleotide prior to conversion; (b) amplifying said nucleic acid molecule treated with said agent; (c) real-time sequencing said amplified nucleic acid molecule; and (d) detecting whether said nucleotide is formerly methylated or not methylated in said predetermined position in the sample wherein a methylated or not methylated nucleotide is indicative of a pathological condition or the predisposition for said pathological condition.

This work was supported by grants 01SF9902/0 and 01KW9967 of the Federal Department of Education and Research of Germany.

In this specification, a number of documents is cited. The disclosure content of these documents, including manufacturers' manuals, is herewith incorporated by reference.

### BACKGROUND OF THE INVENTION

Methylation of nucleotides such as CpG dinucleotides or methylated adenine or guanine residues on the DNA but also on the RNA level is a key element of the epigenetic control of genomic information in mammals [1]. It plays a crucial role in chromatin structure and gene expression, and aberrant DNA methylation, including hypo- as well as hypermethylation, is often associated with pathogenesis, such as tumorigenesis [2]. Multilocus methylation profiles can make tumor types distinguishable [3] or can elucidate a distinct subgroup within a histologically indistinguishable tumor panel [4]. Differences in methylation profiles can be of prognostic value [5]. Minimal traces of aberrant methylated DNA fragments in blood serum could serve as early diagnostic markers [6]. Although a variety of methods are available to assess the methylation status in biological material, studying methylation is still limited by the low sensitivity and/or the high consumption of time and labor of current protocols. Restriction enzyme-based. Techniques often require large amounts of DNA and the loci, which can be investigated, are restricted to recognition sites of the enzymes [3,4]. Sodium bisulfite-treatment of DNA converts unmethylated cytosine into uracil, which is subsequently amplificated as thymine in a PCR. Methylated cytosine, however, is non-reactive and remains detectable as a cytosine. On bisulfite-converted DNA several techniques have been applied to assess the methylation status. These either suffer from a low throughput [7,8] or a labor intensive experimental set up [9,10] and/or very low sensitivity and inaccurate quantitation [7, 8, 9, 10], and/or are limited to the detection of only one distinct nucleotide in one reaction [8, 9]. What is needed in the art is a method that overcomes the limitations mentioned above.

### SUMMARY OF THE INVENTION

The present invention relates to a method for the detection of the methylation status of a nucleotide at a predetermined position in a nucleic acid molecule comprising the steps of (a) treating a sample comprising said nucleic acid molecule or consisting of said nucleic acid molecule in an aqueous solution with an agent suitable for the conversion of said nucleotide if present in (i) methylated form; or (ii) non-methylated form to pair with a nucleotide normally not pairing with said nucleotide prior to conversion; (b) amplifying said nucleic acid molecule treated with said agent; (c) real-time sequencing said amplified nucleic acid molecule; and (d) detecting whether said nucleotide is formerly methylated or not methylated in said predetermined position in the sample. The invention further relates to a method for the diagnosis of a pathological condition or the predisposition for a pathological condition comprising detection of a methylation status nucleotide at a predetermined position in a nucleic acid molecule comprising the steps of (a) treating a sample comprising said nucleic acid molecule or consisting of said nucleic acid molecule in an aqueous solution with an agent suitable for the conversion of said nucleotide if present in (i) methylated form; or (ii) non-methylated form to pair with a nucleotide normally not pairing with said nucleotide prior to conversion; (b) amplifying said nucleic acid molecule treated with said agent; (c) real-time sequencing said amplified nucleic acid molecule; and (d) detecting whether said nucleotide is methylated or not methylated in said predetermined position in the sample wherein a methylated or not methylated nucleotide is indicative of a pathological condition or the predisposition for said pathological condition. The term “methylated” in step (d) refers to the state of the nucleotide before step (a); therefore, in step (d), the nucleotide may no longer be methylated. However, the detection of step (d) allows a conclusion as to whether the nucleotide in a given position was methylated before the application of step (a). The description of step (d) may thus also refer to “detecting whether said nucleotide is formerly methylated”. The inclusion of the adjective “formerly” merely relates to the fact that the nucleotide may change its methylation status in step (a).

The figures show:

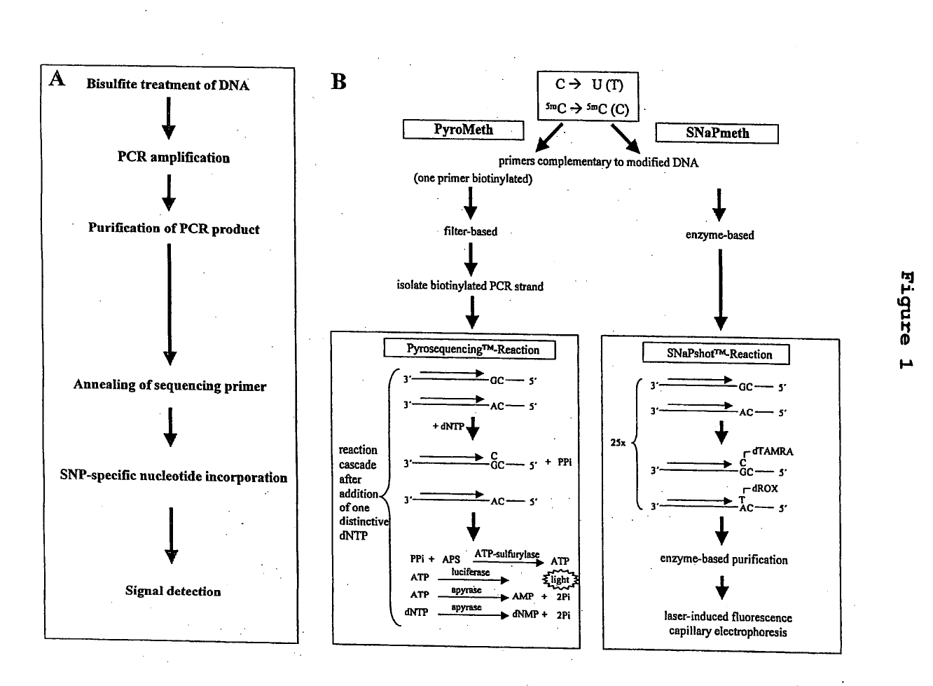

FIG. 1. Schematic representation of the experimental approach used to quantitate the methylation grade at a distinct CpG by Pyrosequencing (PyroMeth). (A) shows an outline of the principal steps of the system. (B) gives a more detailed overview of the method-specific steps required for the sample preparation and sample analysis. The method allows the detection of the nucleotide incorporation at the MethylSNP site in real-time.

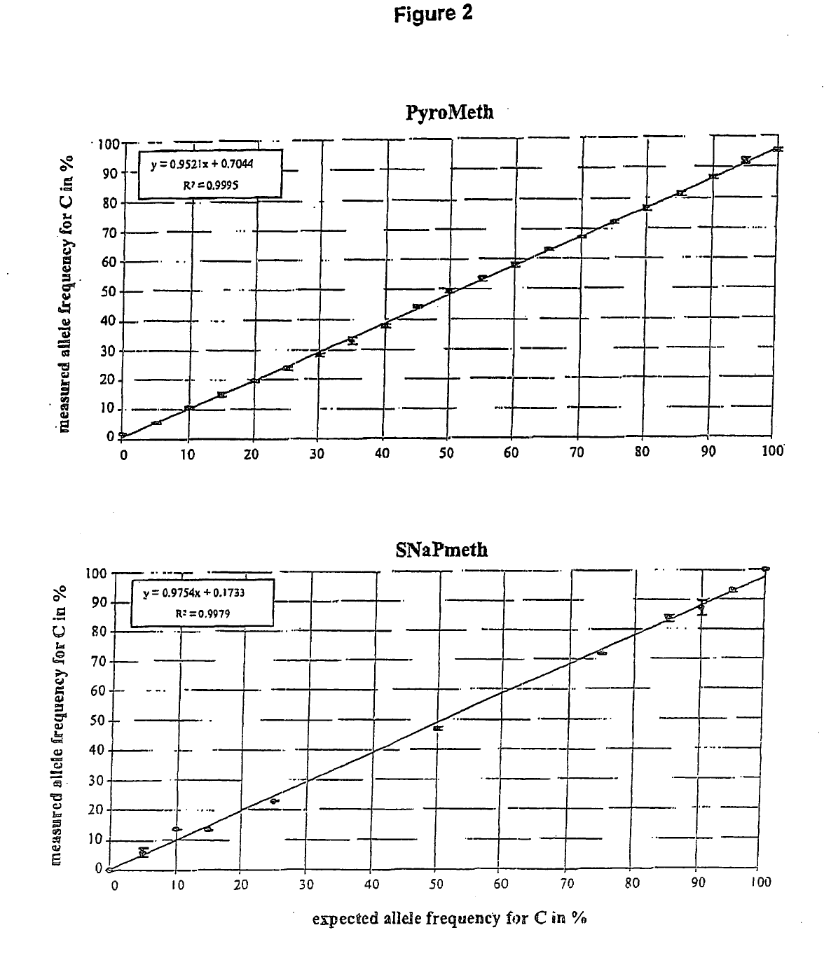

FIG. 2. Calibration plot for determination of allele frequencies in a sample pool with PyroMeth. PCR products, homozygous for either C or T, where mixed in different proportions, in which allele C represents a methylated and T an unmethylated CpG. The measured allele frequency is plotted against the expected allele frequency as defined by the ratios of the two PCR products mixed together, Allele frequencies were calculated using the peak heights % C=peak C/(peak C+peak T)×100. A linear relationship over the whole range of tested allele frequencies could be confirmed (R²=0.9995). For each datapoint four independent analyses were performed (SD are indicated by vertical bars). The standard linear regression formulars were used later on to normalize the data of patient and control samples.

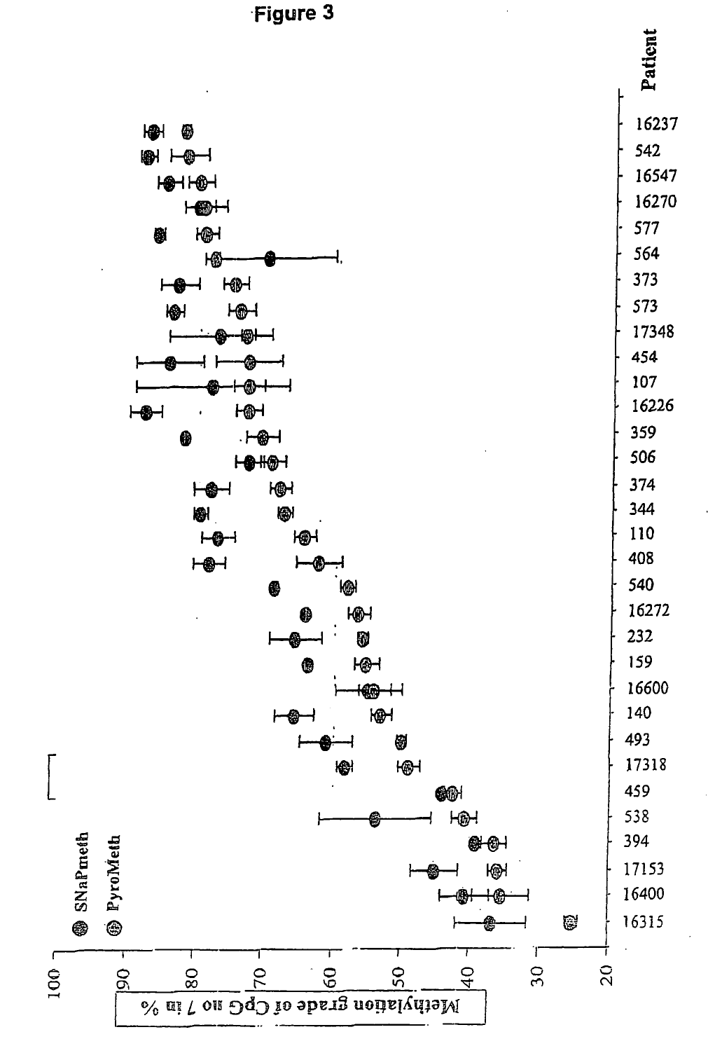

FIG. 3. Comparison of data obtained with the two different MethylSNP analysis techniques SNaPmeth and PyroMeth. Only results for the pilocytic astrocytoma tumor subtype are shown (n=32). The data are sorted by an increasing methylation grade as obtained with PyroMeth (grey circles). Black circles represent values obtained with SNaPmeth. For each data point two individual PCR reactions were carried out and either analysed once (SNaPmeth) or twice each (SD are indicated by vertical bars).

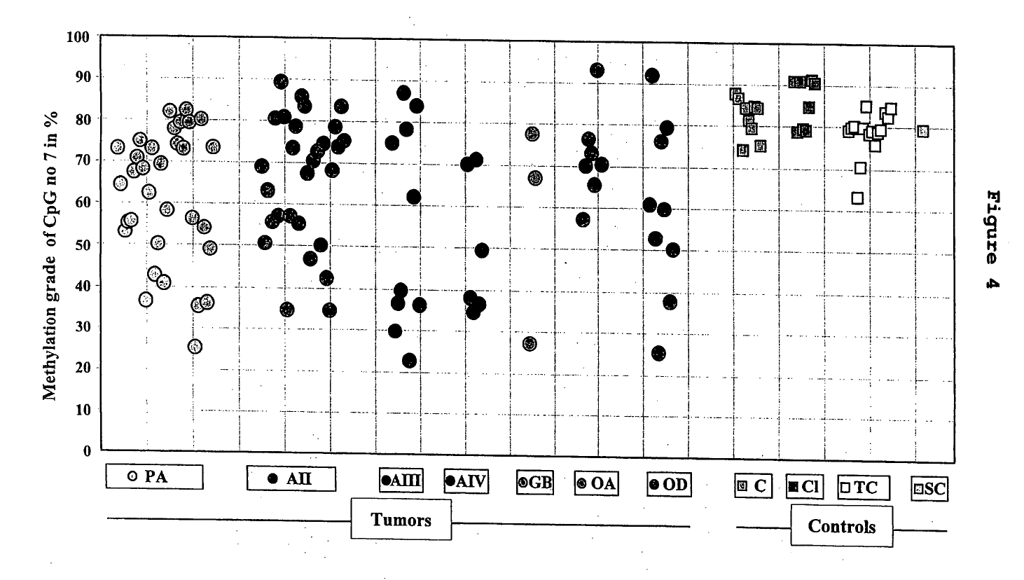

FIG. 4. Methylation analysis of CpG no 7 with PyroMeth. 95 tumors and 33 controls could be analysed successfully. Every circle and square represents an individual sample. The colour code of the sample groups are given below the diagram. All tumor group show an indistinguishably broad range of methylation (22%-93%), regardless of their WHO grade and subtype. In contrast, normal brain tissues and spinal cord seem to be consistently highly methylated (range from 63% to 91%). Thus, many of the tumor samples are hypomethylated. PA, pilocytic astrocytoma; AII, low grade astrocytoma; AIII, anaplastic astrocytoma; AIV, secondary glioblastoma; GB, primary glioblastom; OD, oligodendroglioma; OA, oligoastrocytoma; C, cerebrum; CI, cerebellum; TC, truncus cerebri; SC, spinal cord.

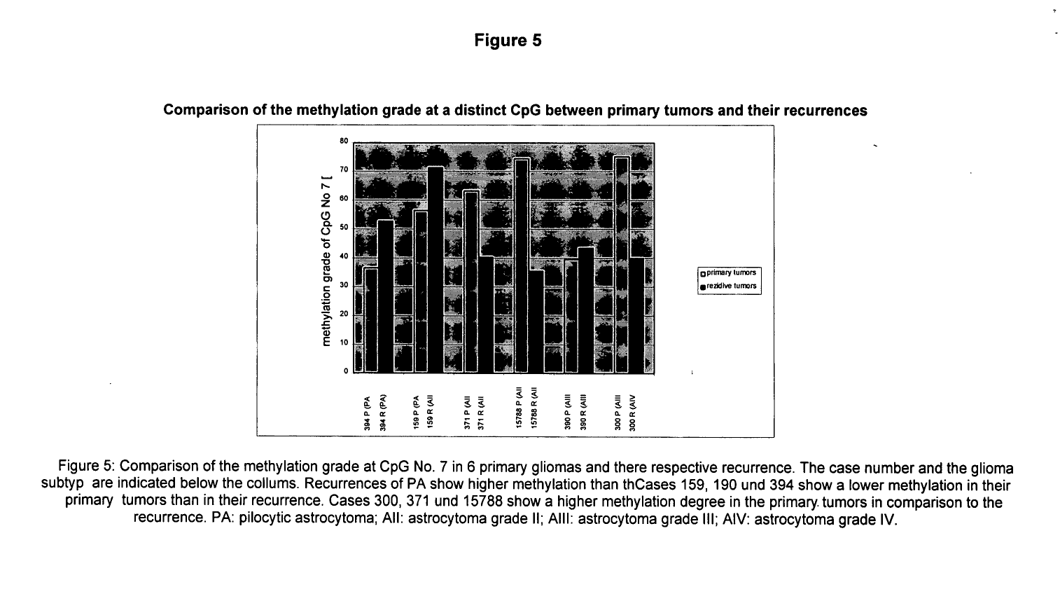

FIG. 5. Comparison of the methylation grade at CpG No. 7 in 6 primary gliomas and there respective recurrence. The case number and the glioma subtype are indicated below the collums. Recurrences of PA show higher methylation than the primary tumors. Recurrences of AII show a lower methylation in their recurrence than in the primary tumors. The methylation grade therefore makes the glioma subtypes and there recurrences distinguishable. PA: pilocytic astrocytoma; AII: astrocytoma grade II; AIII: astrocytoma grade III; AIV: astrocytoma grade IV.

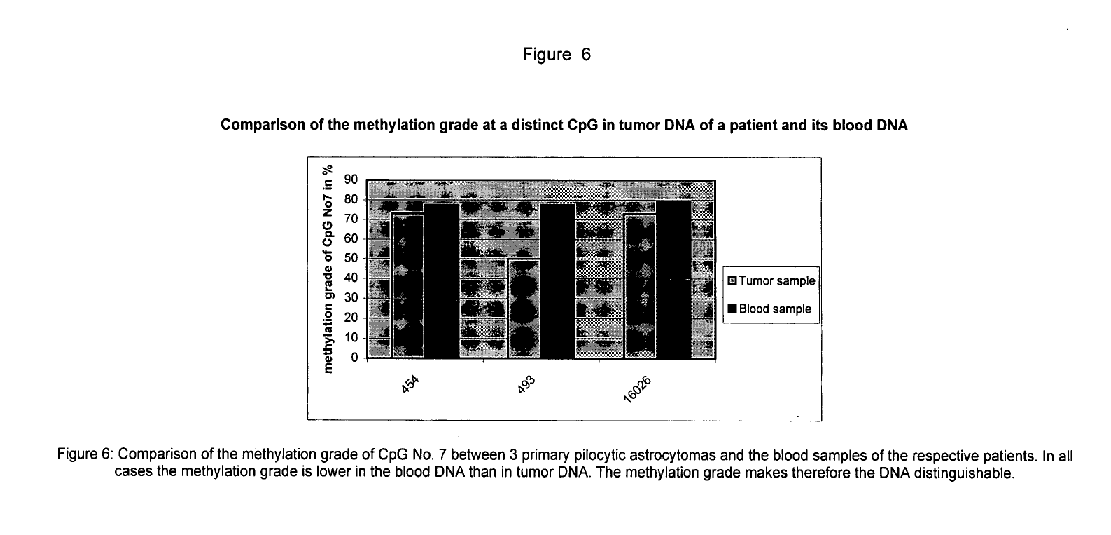

FIG. 6. Comparison of the methylation grade of CpG No. 7 between 3 primary pilocytic astrocytomas and the blood samples of the respective patients. In all cases the methylation grade is lower in the blood DNA than in tumor DNA. The methylation grade makes therefore the DNA distinguishable.

### DETAILED DESCRIPTION OF THE INVENTION

The present invention relates to a method for the detection of a methylated nucleotide at at least one predetermined position in a nucleic acid molecule comprising the steps of (a) treating a sample comprising said nucleic acid molecule or consisting of said nucleic acid molecule in an aqueous solution with an agent suitable for the conversion of said nucleotide to pair with a nucleotide normally not pairing with said nucleotide; (b) amplifying said nucleic acid molecule treated with said agent; (c) real-time sequencing said amplified nucleic acid molecule; and (d) detecting nucleotides that formerly methylated or not methylated in said predetermined position in the sample.

The term “methylated nucleotide” refers to nucleotides that carry a methyl group attached to a position of a nucleotide that is accessible for methylation. As has been detailed herein above, these methylated nucleotides are found in nature and are often used as epigenetic markers. The most important example to date is methylated cytosine that occurs mostly in the context of the dinucleotide CpG, but also in the context of CpNpG- and CpNpN-sequences. In principle, other naturally occurring nucleotides may also be methylated.

The term “predetermined position in a nucleic acid molecule” is used in correction with the fact/denotes the fact that in a predetermined specific position within the nucleic acid molecule, it is known which type of nucleotide (adenine, cytosine or guanine) is present. Advantageously, the nucleotide sequence around this nucleotide is also known. Such knowledge may be derived from prior established sequencing data such as from databases. It is advantageously also known that the nucleotide in this position may occur in a methylated or in a non-methylated state, depending on the status of the cell or tissue harboring said nucleotide. The state may be associated or indicative of, for example, a disease or a differentiation status. Methylation appears, at least in some cases, to be reversible. Analysis of the methylation status of the nucleotide in the predetermined position will in many cases, with a high degree of reliability, allow a conclusion with regards to the status of the cell or tissue. Simultaneously, often a conclusion may be drawn with respect to, for example, the disease state of the organism, such as a mammal and most preferably a human from which this cell originates.

The term “sample” means, in connection with the present invention, any sample of natural or non-natural origin that carries a nucleic acid molecule. The nucleic acid molecule may also be either of natural or non-natural origin. It may be single- or double-stranded and includes oligo- as well as polynucleotides. It is preferred that the sample is of natural origin. It is further preferred that the sample is derived from a mammal, preferably a human. Preferred are further those embodiments, wherein said sample is derived from a tissue, a body fluid or stool. A tissue sample is any sample that may be taken from a vertebrate and preferably a mammal for analysis. If a tissue sample or other sample is taken from a human, the human will have to give his informed consent. Tissue samples include those from skin, muscle, cartilage, bone or inner organs such as liver, heart, kidney, brain, nerve tissue, spleen, pancreas, gut and stomach wherein this list is not be understood as exclusive. Body fluids include blood and fluids derived therefrom such as serum, urine, intestinal fluid and sputum, to name the most important ones.

The term “comprising said nucleic acid molecule or consisting of said nucleic acid molecule in an aqueous solution” describes, in connection with the present invention, the options that the sample may comprise the nucleic acid molecule to be analysed alone or together with other components that may occur in the neighborhood of the nucleic acid molecule in its natural state such as components derived from a cell. Examples of such components are RNAs such as rRNA or mRNA when DNA is under investigation or residual genomic or plasmid DNA when RNA is to be analysed. Care should be taken to remove components from the sample that can interfere with the desired analysis. Alternatively, the sample may consist of the nucleic acid molecule in an aqueous solution. This alternative requires that the nucleic acid molecule after synthesis or extraction from a cell has substantially been purified. It is important to note that in this regard, the term “consisting of” also encompasses the term “essentially consisting of” In accordance with the present invention. The nucleic acid molecule is thus at least 95%, preferably at least 98%, more preferred at least 99% and most preferred at least 99.8% pure, irrespective of the contents of the aqueous solution. The aqueous solution may be water such as distilled water, a buffered solution such as a phosphate buffered solution or buffered solution other than a phosphate buffered solution, to name some important examples. It is mandatory that the solution is free or essentially free of enzymes that unspecifically degrade the nucleic acid molecule to be analysed such as DNases or RNases. On the other hand, the method of the invention, if desired, may be carried out in the presence of specifically degrading enzymes such as restriction enzymes or ribozymes. For certain purposes, small interfering RNAs may also be tolerated.

The term “agent suitable for the conversion of said nucleotide if present in (i) methylated form or (ii) non-methylated form to pair with a nucleotide normally not pairing with said nucleotide prior to conversion” refers to an agent such as sodium bisulfite (sodium hydrogen sulfite, (NaHSO₃) and hydrochinone 1,4-dihydroxybenzene (C₆H₆O₂)) that converts a cytosine nucleotide in its (in this case:) non-methylated state (in other cases: methylated state) into uracil (for other nucleotides other conversion products are feasible) so that it pairs with a adenosine instead of a guanine. Upon the subsequent generation of the complementary strand, an adenine will be inserted instead of a guanine thus giving rise to an SNP (“MethSNP”) in this position. Similarly, adenine may be converted by nitric acid (HNO₃) to hypoxanthine to give rise to a nucleotide pairing with cytosine, whereas guanine can be treated with ethylmethanesulfonate to give rise to a nucleotide pairing with thymine. Insofar, the normal Watson-Crick pairing in this predefined position is maintained (adenine:thymidine/uracil and cytosine:guanine) upon subsequent amplification of the strands but a different nucleotide pair may be present, depending upon the methylation status of the nucleotide originally present in this position. It is to be understood that the aforementioned agents are not intended to limit the invention but are preferred examples of possible agents. Included within the scope of the invention are agents that convert the non-methylated or the methylated nucleotide as mentioned hereinabove. Preferred are agents that convert nucleotides in the non-methylated state.

The term “amplifying” refers to any method that allows the generation of a multitude of identical or essentially identical (i.e. at least 95% more preferred at least 98%, even more preferred at least 99% and most preferred at least 99.5% such as 99.9% identical) nucleic acid molecules or parts thereof. Such methods are well established in the art; see Sambrook et al. “Molecular Cloning, A Laboratory Manual”, 2^{nd} edition 1989, CSH Press, Cold Spring Harbor. They include polymerase chain reaction (PCR) and modifications thereof, ligase chain reaction (LCR) to name some preferred amplification methods.

The term “real-time sequencing” denotes, in accordance with the present invention sequence analyses which allow specific sequencing, i.e. determination of the sequence of a nucleic acid molecule in real-time. Real-time sequencing allows to immediately monitor the incorporation of nucleotides by polymerases such as, for example, DNA or RNA polymerases by either fluorescence or luminescence signals which are subsequently emitted. Real-time sequencing techniques include but are not limited to Pyrosequencing or fluorescence didesoxy nucleotide sequencing.

The detection step may be any suitable detection step that can differentiate between a methylated and a corresponding non-methylated nucleotide. A preferred detection method is described herein below. The detection step of the luminescence or fluorescence signal may be any suitable detection step that can differentiate between a formerly methylated (e.g. after conversion C) and a corresponding formerly non-methylated nucleotide (e.g. after convertion T). The preferred detection method is Pyrosequencing. This method is based on the release of inorganic pyrophosphate (PPi) when a nucleotide has been incorporated in a growing nucleic acid strand during a polymerase reaction. The released pyrophosphat can be detected after a enzymatically driven reaction which subsequently generate light. The amount of the generated light is proportional of the amount of nucleotides incorporated as for each incorporated nucleotide PPi is released and can initiate the above described reaction cascade. Formerly methylated and formerly non-methylated nucleotides can be dicriminated and their repective amount can be quantitated through the distinct amounts of generated light when distinct nucleotides were incorporated.

The method of the invention overcomes the above mentioned deficiencies of the prior art methods. In its simples aspect, the method of the invention combines treatment of nucleic acids with the aforementioned agent so to generate new pairing partners upon subsequent amplification, amplification and real-time sequencing to a novel combination of steps neither envisages nor suggested by the prior art. Importantly, the method of the invention is amenable to high throughput (HTS) analysis. For example said treatment of the nucleic acid with the aforementioned agent for converting said nucleotides can be carried out automatically by robots, with e.g. capillary devices and in parallel, e.g. in microtiter plates, to treat a great number of samples in parallel. All steps of the amplification reaction and detection of the formerly methylated or non-methylated nucleotide in said samples are carried out in microtiterplates by robots in the high throughput format.

In one sequencing reaction up to 30 bp or more can be investigated. Therefor not only one but if preferred all nucleotides formerly methylated or not methylated in this said sequence can be detected. For example, in one microtiter plate, for example, 96 different gene loci can be screened for methylated/nonmethylated cytosine nucleotides. By applying the above-mentioned methods, for example, 3 CpG dinucleotides in their methylated/non-methylated status can be detected. Accordingly, in case of 96 different gen loci, up to 288 CpG dinucleotide can be detected. It is also envisaged, that the above-mentioned methods are applied in multiplex format. Thus, the present invention facilitates the quantitation of methylated versus non-methylated nucleotides at the respective positions. Namely, the amount of the emitted light during a real-time sequencing of the respective gene focus is proportional to the amount of incorporated nucleotides at the respective position. PyroMeth software calculates the frequency of alleles, i.e. methylated/non-methylated nucleotides on the basis of the emitted light. Particularly, allele frequencies were calculated using the peak heights: % C=peak C/(peak C+peak T)×100.

In another preferred embodiment of the method of the invention, said tissue is a tumor tissue, a tissue affected by a neurodegenerative disease or a tissue affected with another neurological disorder. More preferred tumors which may be analysed in accordance with the invention include primary tumors, metastases or residual tumors. Neurological or neurodegenerytic diseases/disorders comprise diseases/disorders affecting the brain or the central nervous system leading to, for example, failures of the brain and/or nervous systems. It is also envisaged that the method of the invention can be used to analyse immune deficiencies or growth abnormalities.

The method in a particularly preferred embodiment considers that said primary tumor is a glioma. Additionally, in another particularly preferred embodiment said primary tumor is a solid tumor such of the skin, breast, brain, cervical carcinomas, testicular carcinomas, etc. More particularly, cancers that may be diagnosed by using the methods of the present invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocarbilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. It is most preferred that said glioma is an astrocytoma, an oligodendroglioma, an oligoastrocytoma, a glioblastoma, a pilocytic astrocytoma. Examples of astrocytomas that may be analysed in accordance with the invention include those mentioned in the appended examples.

Also preferred are embodiments wherein said neurodegenerative disease is Alzheimer disease, Parkinson disease, Huntington disease, Rett-Syndrome.

As regards said further neurological disorders it is preferred that those are Prader-Willi-Syndrome or Angelman-Syndrome, Fragile X-Syndrome, ATR-X-Syndrome.

The nucleic acid molecule may be any nucleic acid molecule known in the art including a peptide nucleic acid molecule (PNA). In an additional preferred embodiment of the method of the invention said nucleic acid molecule is a DNA molecule or an RNA molecule. DNA molecules include genomic DNA as well as cDNA wherein genomic DNA is preferred since naturally occurring. RNA includes ribosomal RNA (rRNA), transfer RNA and messenger RNA (mRNA). Again, it is to be understood that these options are preferred options that are not intended to limit the scope of the invention.

As mentioned above, a variety of amplification methods are known in the art, all of which are expected to be useful in the method of the invention. It is preferred that the amplification in step (b) is effected by LCR or PCR. The PCR is a powerful technique used to amplify DNA millions of fold, by repeated replication of a template. In a short period of time. The process utilizes sets of specific in vitro synthesized oligonucleotides to prime DNA synthesis. The design of the primers is dependent upon the sequences of the DNA that is desired to be analyzed. It is known that the length of a primer results from different parameters (Gillam (1979), Gene 8, 81-97; Innis (1990), PCR Protocols: A guide to methods and applications, Academic Press, San Diego, USA). Preferably, the primer should only hybridize or bind to a specific region of a target nucleotide sequence. The length of a primer that statistically hybridizes only to one region of a target nucleotide sequence can be calculated by the following formula: (¼)^{x} (whereby x is the length of the primer). For example a hepta- or octanucleotide would be sufficient to bind statistically only once on a sequence of 37 kb. However, it is known that a primer exactly matching to a complementary template strand must be at least 9 base pairs in length, otherwise no stable-double strand can be generated (Goulian (1973), Biochemistry 12, 2893-2901). It is also envisaged that computer-based algorithms can be used to design primers capable of amplifying the nucleic acid molecules of the invention. Preferably, the primers of the invention are at least 10 nucleotides in length, more preferred at least 12 nucleotides in length, even more preferred at least 15 nucleotides in length, particularly preferred at least 18 nucleotides in length, even more particularly preferred at least 20 nucleotides in length and most preferably at least 25 nucleotides in length. The invention, however, can also be carried out with primers which are shorter or longer.

The PCR technique is carried out through many cycles (usually 20-50) of melting the template at high temperature, allowing the primers to anneal to complimentary sequences within the template and then replicating the template with DNA polymerase. The process has been automated with the use of thermostable DNA polymerases isolated from bacteria that grow in thermal vents in the ocean or hot springs. During the first round of replication a single copy of DNA is converted to two copies and so on resulting in an exponential increase in the number of copies of the sequences targeted by the primers. After just 20 cycles a single copy of DNA is amplified over 2,000,000 fold.

The LCR is another technique that allows detection of single point mutations in disease genes (Taylor (1995), Curr Opin Biotechnol. 1, 24-29; Yamanishi (1993), Hum Cell 2, 143-147; Laffler (1993) Ann Biol Clin 51, 821-6). The technique utilizes a thermostable DNA ligase to ligate together perfectly adjacent oligos. Two sets of oligos are designed to anneal to one strand of the gene at the site of the mutation, a second set of two oligos anneals to the other strand. The oligos are designed such that they will only completely anneal to the wild-type sequences. In the example shown below for the sickle-cell mutation, the 3′ nucleotide of one oligo in each pair is mismatched. This mismatch prevent the annealing of the oligos directly adjacent to each other. Therefore, DNA ligase will not ligate the two oligos of each pair together. With the wild-type sequence the oligo pairs that are ligated together become targets for annealing the oligos and, therefore, result in an exponential amplification of the wild-type target. Given that prior sequence knowledge is required in order to detect point mutations in disease genes, the LCR technique is utilized for the diagnosis of the presence of a mutant allele in high risk patients.

For further guidance, see Taylor (1995), Curr Opin Biotechnol. 1, 24-29, Yamanishi (1993), Hum Cell 2, 143-147, and Laffler (1993) Ann Biol Clin 51, 821-6.

In a particularly preferred embodiment of the method of the invention, one amplification primer is detectably labeled. The detectable label advantageously forms an anchor which allows removal of single stranded amplified molecules after amplification. In a further particularly preferred embodiment of the invention, said amplification products are therefore converted into single stranded molecules (e.g. upon heat application such as application of temperatures higher than 90° C.) prior to further processing in step (c). The anchor may be taken up by a further molecule which may be affixed to a solid support such as a chip, a bead, a column material, a microtiter plate etc. having a glass surface, a plastic surface such as a homopolymer or on other surface. The anchor and further molecule may be binding pair that have naturally a high affinity exceeding 10e6 M such as an antibody/antigen pair or a biotin/avidin or biotin/streptavidin pair.

In accordance with the present invention, it is especially preferred that said label is biotin, avidin, streptavidin or a derivative thereof or a magnetic bead. Derivatives of streptavidin include molecules having a lower binding affinity for biotin and include Strep-tag I, Strep-tag II and Strep-tag III described in DE-A1 101 13 776 or U.S. Pat. No. 5,506,121. These molecules allow the dissociation from biotin under rather mild physiological conditions.

It is also preferred in accordance with the present invention that said methylated nucleotide is an adenine, guanine or a cytosine.

In a most preferred embodiment of the method of the present invention, said real-time sequencing comprises:
(a) hybridization of a sequencing primer to said amplified nucleic acid molecule in single-stranded form;
(b) addition of a DNA polymerase, a ATP sulfurylase, a luciferase, an apyrase, adenosine-phosphosulfate (APS) and luciferin;
(c) sequential addition of all four different dNTPs;
(d) detection of a luminescent signal wherein the intensity of the luminescent signal is correlated with the incorporation of a specific nucleotide at a specific position in the nucleic acid molecule and wherein the intensity of said signal is indicative of the methylation status of said nucleotide in said predetermined position.

Most preferably, real-time sequencing is performed by the pyrosequencing method which is further explained in the appended examples. In said pyrosequencing method an amplified nucleic acid molecule is separated to a single-stranded nucleic acid molecule, as described herein below. The DNA complementary strand synthesis is subsequently done after annealing of a further primer monitored to determine sequences, namely, pyrophosphate released as a reaction product upon synthesizing a complementary DNA strand is converted into ATP, which reacts with luciferine using luciferase to generate luminescence. Since pyrosequencing is inexpensive and can be used, e.g., for sequencing a large number of samples simultaneously, it is applicable as a high throughput monitor for DNA. Pyrosequencing is briefly explained as follows. The apparatus used is a so-called luminescence photometer. Reagents, including DNA samples; primers to determine the starting point of complementary strand synthesis; DNA synthesizing enzymes; an enzyme a pyrase to decompose dNTP which has been added as a substrate and remained unreacted; sulfurylase to convert pyrophosphate into ATP; luciferine; and luciferase involved in the reaction of luciferine with ATP, are placed in a titer plate. At this moment, no complementary strand synthesis occurs because dNTP, a substrate for the reaction, is not present. Four kinds of dNTP (i.e., dATP, dCTP, dTTP and dGTP) are added in a designated order from the top of the reaction vessel, for example, by an ink jet system. If dCTP is the designated base to be synthesized, no reaction occurs when dATP, dTTP or dGTP is added. Reaction occurs only when dCTP is added, then the complementary strand is extended by one base length, and pyrophosphate (PPi) is released. This pyrophosphate is converted into ATP by ATP sulfurylase and the ATP reacts with luciferine in the presence of luciferase to emit chemiluminescence. This chemiluminescence is detected using a secondary photon multiplier tube or the like. Remaining dCTP or unreacted dNTP is decomposed by apyrase which converts it into a form which has no effect on the subsequent repetitive dNTP injection and the reaction which follows. The four kinds of dNTPs are added repeatedly in a designated order and the base sequence is determined one by one according to the presence or absence of chemiluminescence emitted each time. (see Ronaghi, M. et al. Science 281, 363-365 (1998)). The reported possible length of DNA to be sequenced ranges between 20 bases and 30 bases, however, is not limited thereto. This is because the sequencing is involved in a step reaction, in which the efficiency of the reaction is largely affected by the possible length of the base to be sequenced. Examples of possible systems in which pyrosequencing is used include a palm-sized DNA sequencer, a DNA sequencer for large scale analyses for gene diagnosis or comparative analyses, and a DNA mutation analysis system.

Further, various primers can be immobilized on a solid surface, beads or the like, and the target nucleic acid is obtained by hybridizing a double-stranded nucleic acid sample with these primers so that a necessary and sufficient amount of nucleic acid sample can be readily supplied. Since the target nucleic acid can be injected into a reaction vessel without processing it into a single strand, only a simple sample preparation is required for the sequencing reaction.

Longer DNAs can also be sequenced and analyzed by carrying out a sufficient and thorough reaction. Therefore, the structure of the reaction vessel is devised such that the reaction chamber is in contact with a vibrating element to thoroughly mix added dNTP with a reaction solution. The reaction efficiency can be increased by stirring the injected dNTP.

In the DNA base sequencing method, pyrophosphate produced upon a DNA complementary strand synthesis is converted into ATP, the ATP is reacted with luciferine using luciferase to generate chemiluminescence, the emitted chemiluminescence is detracted, whereby the kind of incorporated nucleic acid is detected and thus the base sequence is determined. The four kinds of dNTPs are supplied into a reaction vessel in a designated order by pressurizing via capillaries or narrow grooves which connect the reaction vessel and reagent reservoirs. Also a palm-sized DNA sequencing apparatus can be used, and many kinds of DNAs can be simultaneously analyzed by providing a multiple number of reaction chambers in a small area.

It is also preferred in accordance with the invention that the method further comprises quantifying the methylated nucleotides (or, alternatively non-methylated nucleotides).

The quantification of methylated nucleotides is an important means to draw in many instances a valid conclusion with regard to the epigenetic status of the analysed sample, for example, the methylation grade of nucleotides allows to draw conclusions about the progression of tumors or allows to draw conclusions about the response of an individual during therapy or allows to distinguish normal tissue from tumor tissue. This is because the analysed tissues or cell samples may not be uniformly methylated or not methylated in a specific predefined chromosomal position. Rather, a majority of cells only may be methylated or not methylated in said specific predefined chromosomal position. Insofar, a quantitation of the readout will help in providing a meaningful analysis. Quantitation is best carried out by including an internal standard such as a tissue or cell sample known to consist or essentially consist (with regard to the percent values in connection with the term “essentially”, see above) of methylated or non-methylated nucleotides at the position of interest. Alternatively an recombinantly or artificial ((semi)synthetically produced) nucleic acid molecule may serve as a control. The skilled artisan may without undue burden determine conditions or use host cells that are devoid of a methylation system. Alternatively, the nucleic acids may be methylated within a cell or in vitro using appropriate methylases. As mentioned hereinabove, quantitation is done by analysing the emitted light arising due to incorporation of nucleotides.

The method of the invention in a different preferred embodiment requires that said agent suitable for the conversion of said nucleotide to pair with a nucleotide normally not pairing with said nucleotide is sodium bisulfite.

As stated elsewhere in this specification, bisulfite reacts with non-methylated cytosine and changes its base-pairing behaviour. After bisulfite treatment, the former cytosine residue (now an uracil) pairs after subsequently amplified as a thymine in an amplifying reaction with adenine.

A particularly preferred version of this embodiment is further detailed below:

As mentioned, this embodiment takes advantage of the fact that bisulfite modification of genomic DNA creates common single nucleotide polymorphisms (SNPs), such as [C/T], at differentially methylated CpGs, which we call MethylSNPs. On the one hand, the primer extension approach SNaPshot™ from Applied Biosystems, to investigate a particular MethylSNP was used, calling this version SNaPmeth. This approach was compared with the method of the present invention, which makes in this specific embodiment use of the sequencing-by-synthesis technique Pyrosequencing™ from Pyrosequencing to analyse the percentage of methylation at the same CpG. This embodiment of the invention is also called technique PyroMeth (FIG. 1). In SNaPmeth, the polymerase extends a primer complementary to the bisulfite-modified DNA template by adding only a single fluorescently labeled nucleotide to its 3′ end. After capillary electrophoresis of the extended primer, the labeling of the four dideoxynucleotide triphospates (ddNTPs) with different fluorescent dyes allows the GeneScan® software to distinguish between the two bases incorporated at the polymorphic site. In PyroMeth, the MethylSNP is analysed by real-time sequencing, based on the detection of the stepwise nucleotide incorporation by luminescence. After hybridization of primer and template, the four deoxynucleotide triphosphates (dNTPs) are added separately according to a predetermined dispensation order. Only if the offered nucleotide is complementary to the bisulfite-treated template is it incorporated and inorganic pyrophospate (PPi) is released. PPi drives an ensuing reaction cascade at the end of which a certain amount of light is released that is equivalent to the amount of incorporated nucleotides. Unincorporated dNTPs are degraded after each reaction cycle and therefore the intensity of any light signal can be reliably assigned to a specific dNTP. We used both methods to test methylation of CpG no 7 [11] in 97 primary tumors of different glioma subtypes and 33 control tissues derived from three parts of the brain and spinal cord as a biomarker for molecular diagnosis of pilocytic astrocytomas. As is evident from the appended example, the method of the invention is superior to the SnaPmeth technology.

The invention further relates to a method for the diagnosis of a pathological condition or the predisposition for a pathological condition comprising detection of the methylation status of a nucleotide at at least one predetermined position in a nucleic acid molecule comprising the steps of (a) treating a sample comprising said nucleic acid molecule or consisting of said nucleic acid molecule in an aqueous solution with an agent suitable for the conversion of said nucleotide if present in (i) methylated form; or (ii) non-methylated form to pair with a nucleotide normally not pairing with said nucleotide prior to conversion; (b) amplifying said nucleic acid molecule treated with said agent; (c) real-time sequencing said amplified nucleic acid molecule; and (d) detecting whether said nucleotide is formerly methylated or non-methylated in said predetermined position in the sample wherein a methylated or a not methylated nucleotide is indicative of a pathological condition or the predisposition for said pathological condition.

For the following preferred embodiments, the same definitions and explanations as given herein above for corresponding embodiments apply.

In a preferred embodiment of this method of the invention, said pathological condition is cancer, a neurodegenerative disease or another neurological disorder.

More preferred, said tumor is a primary tumor, a metastasis or a residual tumor. It is particularly preferred that said primary tumor is a glioma and most preferred that said glioma is an astrocytoma, oligodendroglioma, oligoastrocytoma, pilocytic astrocytoma or glioblastoma.

Further preferred in accordance with the method of the invention is that said neurodegenerative disease is Alzheimer disease, Parkinson disease, Huntington disease, Rett-Syndrome.

It is also preferred that said neurological disorder is Prader-Willi-Syndrome or Angelman-Syndrome, Fragile-X-Syndrome, ATR-X-Syndrome.

Again, preferred is further a method wherein said nucleic acid molecule is a DNA molecule or an RNA molecule.

In a different preferred embodiment of this method of the invention, the amplification in step (b) is effected by LCR or PCR. More preferred, amplification is carried out under conditions wherein one amplification primer is detectably labeled. Said label preferably is biotin, avidin, streptavidin or a derivative thereof or a magnetic bead.

Also in this embodiment of the invention, said methylated nucleotide preferably is an adenine, guanine or a cytosine.

It is again particularly preferred that this embodiment of the method of the invention is carried out under conditions wherein said real-time sequencing comprises:
(a) hybridization of a sequencing primer to said amplified DNA in single-stranded form;
(b) addition of a DNA polymerase, a ATP sulfurylase, a luciferase, an apyrase, adenosine-phosphosulfate (APS) and luciferin;
(c) sequential addition of all four different dNTPs;
(d) detection of a luminescent signal wherein the intensity of the luminescent signal is correlated with the incorporation of a specific nucleotide at a specific position in the DNA and wherein the intensity of said signal is indicative of the methylation status of said nucleotide in said predetermined position.

Preferably, the method further comprises steps for quantifying the formerly methylated nucleotides.

Further preferred is, again, that said agent suitable for the conversion of said nucleotide to pair with a nucleotide normally not pairing with said nucleotide is sodium bisulfite.

In all embodiment referred to herein above it is preferred that at least the detection step and more preferred all steps are carried out in the high throughput format. Nucleic acid-extraction from said tissues, body fluid or the like, can be done automatically by robots. Said conversion and purification of said nucleic acids can also be carried out automatically by robots, with e.g. capillary devices and e.g. in microtiterplates. All steps of the amplification reaction and detection of the formerly methylated or non-methylated nucleotide in said samples can be carried out in microtiter plates by robots in the high throughput format.

In a different preferred embodiment, at the same time, the methylation status of more than one predetermined nucleotide such as 2, 3, 4, 5, 6, 7, 8, 9 or 10 or even more, such as at least 20, 50, 100 or 1000 predetermined positions is detected. One sample can be analysed at more than one predetermined nucleotide positions at the same time, or a number of samples can be analysed at more than one predetermined nucleotide positions at the same time. In one detection step of a sample more than one predetermined nucleotide positions can be analysed (multiplexing), either in one nucleic acid fragment, or in 2, 3 or more different nucleic acid fragments.

The present invention, thus generally described, will be understood more readily by reference to the following examples, which are provided by way of illustration and are not intended to be limiting of the present invention.

### EXAMPLE

1 Materials and Methods

1.1 Patient Samples

This study included 97 primary tumor samples, distributed as follows: 32 pilocytic astrocytomas (range 2-35 years, 18 male, 14 female), 29 astrocytomas grade II (range 9-54 years, 12 male, 17 female), 10 astrocytomas grade III (range 3-67 years, 4 male, 6 female), 6 astrocytomas grade IV (range 10-71 years, 3 male, 3 females), 3 glioblastoma multiform (range 46-70 years, 2 male, I female), 7 oligoastrocytomas (range 20-63 years, 2 male, 5 female), 10 oligodendrogliomas (range 17-60 years, 3 male, 7 female). 33 control tissues derived from 9 healthy individuals (range 0.6-88 years, all male) from three parts of the brain, including cerebrum (C, n=9), cerebellum (Cl, n=8) and truncus cerebri (TC, n=15), as well as spinal cord (SC, n=I). Details of the individual patients and specimens are published elsewhere [12]. All tumour and control samples are derived from unrelated patients/individuals. The histological typing of the tissues was done according to the classification of the World Health Organization (WHO) [13]. No substantial contamination of the tumor samples with normal tissues was evident. Control tissues were provided through the Brain and Tissue Bank for Developmental Disorders, University of Miami, USA, contract No. NOI-HD-8-3284 under sponsorship of The National Institutes of Health, except for the samples of individual number 1100 (Department of Pathology, University of Southern California, School of Medicine, Los Angeles, USA) and samples from individual number 7909 (Department of Human Molecular Genetics, Max Planck Institute for Molecular Genetics, Berlin, Germany). All tissues were stored at −20° C. Genomic DNA was extracted following a standard procedure, described elsewhere [14].

1.2 Sodium Bisulfite Conversion

Sodium bisulfite conversion of whole genomic DNA was performed as described previously [15], with slight modifications according to Eads et al. [10]. Briefly, 250 ng of genomic DNA (in a volume of 8 μl) were denatured at 95° C., 10 min, followed by incubation in a 0.3 M NaOH concentrated solution at 42° C., 15 min. DNA and 10 μl of 4% low melting agarose (SeaPlaque, FMC Bioproducts, Rockland, Me., USA) were mixed, and a single bead with a final volume of 20 μl was formed in prechilled mineral oil. Bisulfite conversion was performed with a 5 M sodium bisulfite solution at 50° C., 14 h, under exclusion of light. TE-buffer (pH 8) was used for washing the beads six times, 15 min for each wash. Desulphonation was done in 0.2 M NaOH twice for 15 min each. The second wash with NaOH was neutralized with 1 M HCl, followed by two additional washing steps, again, with TE buffer. For amplification with PCR the agarose beads were diluted with 180 μl HPLC H₂O.

1.3 PCR

Bisulfite converted genomic DNA was amplified with primers. It is preferred that said primers encompass nucleic acid sequences not comprising nucleotides which are formerly methylated. It is also preferred that said primers span regions comprising originally methylated or non-methylated nucleotides. In the present invention, for example, bisulfite converted genomic DNA was amplified with primers fully complementary to the deaminated DNA strands (forward 5′-TGAGTTGGAATAAGTTAGGGTAGATGTG-3′; reverse 5′-CAACTCTCTATATCCCTTTCTAACATAAATCA-3′), yielding a product of 102 bp length. For the PyroMeth assay, the forward primer was biotinylated. The primers do not contain CpG dinucleotides so that the amplification step does not discriminate between templates according to their original methylation status. The following protocol was used for PCR reaction (modifications for the SNaPmeth application are indicated in brackets): the PCR reactions had a total volume of 50 μl (25 μl). 10 μl (5 μl) of agarose-embedded DNA were used as template DNA. The template DNA, 10 μM of each primer, 10 mM dNTPs, 0.4 U Ampli-Taq Gold Polymerase (0.2 U) were incubated with 5.0 μl reaction buffer (2.5 μl). The amplification was performed in a PTC 200 cycler from MJ Research under the program conditions 95° C./10 min followed by 40 cycles of 95° C./1 min, 58° C./1 min, 72° C./1 min, and an extension step at 72° C. for 5 min. For each sample, two independent PCR amplifications were performed and analysed. For PyroMeth, unincorporated primers and dNTPs were separated from the PCR product using the Invisorb PCR HTS 96 Kit (Invitek GmbH, Berlin, Germany). For the SNaPmeth assay, enzyme-based digestion of single stranded oligonucleotides and unicorporated dNTPs was performed, using SAP and Exol according to the supplier's recommendation (Amersham, Braunschweig, Germany).

1.4 Standardization Experiments

To obtain “homozygous” templates for MethylSNP analysis with respect to either the converted CpG or TpG “allele”, cloned PCR fragments of 746 bp length (modified top strand), derived from bisulfite sequencing experiments [11] served as templates for PCR. Amplification products were mixed in different proportions (PyroMeth: 21 proportions in 5% increments from C/T 100:0 to C/T: 0:100). The mean of four independent measurements with standard deviations (SD) were plotted in the calibration plots (FIG. 2). To normalize for background and other factors influencing peak heights and peak areas in a systematic way, data of patient samples and controls were corrected to the calibration curve, according to the calculations outlined below in the MethylSNP analysis section.

1.5 MethylSNP Analysis

SNaPmeth. 1-3 μl of SAP- and ExoI-treated PCR product (˜0.15 pmol) were used for each SNaPmeth primer extension reaction. 0.5 pmol primer (5′-TAGGGGGGTGAATATTGGG-3′) and 1.25 μl SNaPshot Ready Reaction Mix (including AmpliTaq DNA polymerase, fluorescence-labeled (F) ddNTPs, reaction buffer; PE Biosystems, Weiterstadt, Germany) were added to a total reaction volume of 10 μl. Cycling parameters: 96° C./30 sec. 60° C./1 min, 25 cycles in a 96 well microtiter plate. Postextension treatment with SAP (1 h, 37° C.), removed the 5′ phosphoryl groups of unicorporated [F]ddNTPs, prohibiting interference of fluorescence signals during electrophoresis. For electrophoresis on the ABI PRISM® 310 Genetic Analyzer POP-4™ polymer was used, with an injection time 4 sec and a collection time 13 min. The run files were analysed using GeneScan Analysis Software version 2.1. Peak area values were used to calculate allele frequencies in % (e.g. peak area C/peak area C+T)×100), representing the methylation grade at CpG no 7. The mean of the calculated allele frequencies of one sample was normalized to the calibration curve (y=mx+b, with y: observed allele frequency, m: regression coefficient as the slope of the function, x: expected allele frequency, b: intersection point of curve with zero).

PyroMeth. Single-stranded PCR fragments are needed for the sequencing-by-synthesis reaction. To purify the biotinylated PCR fragments they are immobilized on streptavidin-coated Dynabeads® M-280 Streptavidin (Dynal A S, Oslo, Norway), according to the protocol of the SNP Reagent Kit 5×96, Pyrosequencing. After incubation for 15 min at 65° C., the reactions were transferred into a PSQ™ 96 well reaction plate and denatured in 0.5 M NaOH, 10 min. The single stranded PCR fragments were captured with the magnetic rod, transferred in a PSQ 96 well plate and washed in 1× annealing buffer. Again transferred, the single stranded PCR fragments were hybridized with 10 pmol sequencing primer (5′-GGGGTGAATATTGGG-3′) in 1× annealing buffer, 80° C. for 2 min, then moved to room temperature.

The sequencing reaction was performed at 25° C. in a volume of 40 μl 1× annealing buffer on the automated PSQ™ 96 System from Pyrosequencing. Enzyme and substrate from the SNP Reagent Kit were dissolved in each 620 μl high purity water, after reaching room temperature. Then, they were loaded into a special cartridge, just like 160 μl of each deoxynucleoside triphosphate from the same kit. The cartridge and the sample plate were placed into the instrument and the analysis runs automatically. The order of nucleotide dispensation was defined before, corresponding the template sequence. Allele frequencies were calculated using the integrated software SNP Software AQ. Peak heights given in the pyrogram were used to calculate the methylation grade of CpG no 7 in percent (e.g.: % C=peak height C/(peak height C+peak height T)×100). The mean of the calculated allele frequencies of one sample was normalized to the calibration curve (see above).

2 Results

To determine the accuracy of each method for measuring allele frequency within a DNA pool we performed standardization experiments with well-defined DNA samples prior to the analysis of the tumor samples. Since it cannot be guaranteed for any kind of genomic DNA that a CpG of interest is methylated or non-methylated to 100%, we used cloned PCR fragments. These 746-bp fragments were obtained from former bisulfite sequencing experiments [11] and known to comprise our test CpG no 7 either in a “methylated” (CpG) or “unmethylated” (TpG) state. Using the plasmids as templates we generated “homozygous” PCR products containing the test CpG as the only polymorphic site. The amplification products were mixed in different proportions The relationships between peak heights in a pyrogram (PyroMeth) or peak areas in an electropherogram (SNaPmeth) and the underlying allele frequencies were investigated (FIG. 2). A minor allele frequency of 5% could be detected without any problem. The SD obtained with SNaPmeth ranged between 0% and 3.7%, in the PyroMeth assay between 0.2% and 1%. Over the whole data points a linear relationship of the measured allele frequencies was observed. Both, pooling of plasmid DNA and PCR products resulted in satisfying allele frequency detection (data not shown). We used PCR products for the calibration curves to avoid repeated culturing of plasmids and preparation of plasmid DNA.

To investigate the stability of the analysis systems themselves, we assayed twelve individual PCR products two times (data not shown). The PCR products were obtained from genomic DNA from six different patients. For SNaPmeth, the greatest difference in methylation grades, i.e. percent of allele C detected in the same PCR product, was 15.9% with an average difference of 4.7%. For PyroMeth, the greatest difference was 3.6% with an average difference of 0.6%. After determining the accuracy and the reproducibility of SNaPmeth and PyroMeth, we analyzed the methylation grade of CpG no 7 in a total of 97 primary tumor samples of 5 different glioma subtypes. DNA out of 33 tissues from three different parts of the brain and spinal cord served as controls. Data were normalized against the calibration curves as outlined in the materials and methods section. The data obtained with SNaPmeth were very similar to the data generated with PyroMeth. The trends of methylation grades in tumor and control groups were identical. However, the individual methylation values of the samples differed systematically between the two assays. In general, the SNaPmeth assay detected a higher amount of allele C (on average 8%), representing higher methylation (FIG. 3). Overall, the SD for the two independently generated and analysed PCR amplicons showed higher values with this SNP analysis technique (range 0.2%-11.2%, average 3%), as in the real-time sequencing approach PyroMeth (0.5%-4.9%, average 1.8%).

The results about the assessment of CpG no 7 as a potential biomarker are shown in FIG. 4. Data as derived from the sequencing by-synthesis assay PyroMeth are presented. The control groups show a fairly homogeneous methylation in contrast to all tumor subtypes. The latter present a broad range of methylation values irrespective of the tumor grade as shown for astrocytomas. The stratification of the control groups according to the tissue type did not reveal any tissue dependency of methylation. This could be proven for the tumor subtypes as well (data not shown). As methylation may depend on age and gender, we also analyzed the influence of these parameters. Neither age nor gender dependency was found in the tumor samples (data not shown).

3 Discussion

We have described two semi-automated techniques to quantitate DNA methylation at a single CpG. SNaPmeth is based on a single nucleotide primer extension approach with fluorescently labeled ddNTPs while PyroMeth is based on a real-time DNA sequencing technique. Investigating methylation by a primer extension reaction has already been described by others [8]. These authors used radioactively labeled ddNTPs and called their method methylation-sensitive single nucleotide primer extension (Ms-SNuPE). Our non-radioactive version of the method has several advantages: (i) no hazards from radioactivity, (ii) no time consuming pouring, loading, and running of denaturing polyacrylamid gels electrophoresis, (iii) simultaneous detection of C and T alleles in one reaction, and (iv) it is semi-automated and suitable for high-troughput. Thus, SNaPmeth improves this type of approach significantly. If one compares SNaPmeth with PyroMeth, the latter is more reliable and accurate, i.e. the SD values were consistently lower in all experiments.

In total, we screened 130 samples with our new MethylSNP detection methods. As shown in FIG. 3, the trend of methylation found with one approach was nearly exactly mirrored by the results of the other. However, the methylation values determined with SNaPmeth were consistently higher than the values detected with PyroMeth. To allow maximum comparability between the data generated with both methods, the experimental set up was done in parallel as much as possible (e.g. DNA derived from the same bisulfite treatment, one mastermix for all PCRs, same thermocycler). Therefore, we believe that the reason for the slight shift (on average 8%) between the results obtained with both methods lies in the assays themselves. In SNaPmeth, the primer extension reaction occurs in the presence of all four differently fluorescence-labeled ddNTPs, this may lead to a preferential incorporation of particular nucleotides [16]. The competition of nucleotides for incorporation is circumvented in the PyroMeth approach, as the unlabeled dNTPs are added separately one after the other. Furthermore, traces of agarose of the DNA-embedding beads may have a negative influence on capillary ectrophoresis, which has to be carded out for separating the extension products in SNaPmeth [17]. In the PyroMeth approach minimal traces of agarose will be present as well, but they may be better tolerated since no electrophoresis and detection of laser-induced fluorescence is required.

Using the method of two-dimensional (2D) DNA fingerprinting, we previously found CpG no 7 consistently hypomethylated in nearly all pilocytic astrocytomas (10/11) but only a negligible portion of astrocytomas (2/18) under investigation [11]. In this study, we analysed 32 pilocytic astrocytomas and 29 astrocytomas grade II and no difference in methylation of this CpG could be observed between the two glioma subtypes. Rather in both subgroups, a substantial portion of tumor samples was remarkably hypomethylated while others showed the same high level of methylation as the control tissues. This broad range of methylation from 20% to 90% was also found in the other tumor subtypes (FIG. 4). Only among the 7 oligoastrocytomas no dramatic hypomethylation was observed, which might be due to the small number of samples analyzed. Interestingly, we could demonstrate that, among the samples analyzed in this and the previous study, all those with the typical spot shift in 2D DNA fingerprints, indicating the loss of the methyl group at CpG no 7 [11], had a methylation grade below 70%, whereas those without the spot shift had values above 70% (data not shown). It cannot be excluded that tumor samples with a high grade of methylation of CpG no 7 are contaminated with normal tissue but this was not evident from histopathological investigations. Nevertheless, our data confirm that hypomethylation may be as important as hypermethylation in cancer [2]. A database search for coding sequences next to CpG no 7 did not reveal any functional genes in that region. Thus, we no longer consider the observed demethylation of this CpG a pivotal event in tumorigenesis of pilocytic astrocytomas, but an unspectacular concomitant of early tumor development in gliomas.

Any kind of SNP detection method may be adapted for MethylSNP analysis. However, assays suitable for the analysis of allele frequencies in DNA pools, such as Pyrosequencing and SNaPshot, are most appropriate. We have demonstrated that quantitative MethylSNP analysis by the method of the invention is a favorable alternative to existing high-throughput methylation assays [9,10]. Depending on the platform used, between 48 (one-capillary system) and approximately 2,300 genotypes per day (96-capillary system) can be analysed with SNaPmeth. As to PyroMeth, i.e. one preferred embodiment of the invention, the available PTP™ system from Pyrosequencing allows for 25,000 genotypes per day. Thus, with this system a customized panel of 250 CpGs may be analysed in 100 samples within 24 hours.

The invention has been disclosed broadly and illustrated in reference to representative embodiments described above. Those skilled in the art will recognize that various modifications can be made to the present invention without departing from the spirit and scope thereof.

### REFERENCES

1. Robertson, K. D., Wolffe, A. P. (2002) DNA methylation in health and disease. Nat. Rev. Genet, 1, 11-19.
2. Ehrlich, M. (2002) DNA methylation in cancer: too much, but also too little. Oncogene, 21, 5400-5413.
3. Costello, J. F., Fruhwald, M. C., Smiraglia, D. J., Rush, L. J., Robertson, G. P., Gao, X., Wright, F. A., Feramisco, J. D., Peltomaki, P., Lang, J. C., Schuller, D. E., Yu, L., Bloomfield, C. D., Caligiuri, M. A., Yates, A., Nishikawa, R., Su, H. H., Petrelli, N. J., Zhang, X., O'Dorisio, M. S., Held, W. A., Cavenee, W. K., Plass, C. (2000) Aberrant CpG-island methylation has non-random and tumour-type-specific patterns. Nat Genet, 24, 132-138.
4. Toyota, M., Ahuja, N., Ohe-Toyota, M., Herman, J. G., Baylin, S. B., Issa, J. P. (1999) CpG Island methylator phenotype in colorectal cancer. Proc. Natl. Acad. Sci. USA, 96, 8681-8686.
5. Eads, C. A., Lord, R. V., Wickramasinghe, K., Long, T. I., Kurumboor, S. K., Bernstein, L., Peters, J. H., DeMeester, S. R., DeMeester, T. R., Skinner, K. A., Laird, P. W. (2001) Epigenetic patterns in the progression of esophageal adenocarcinoma. Cancer Res., 61, 3410-3418.
6. Usadel, H., Brabender J. Danenberg K D, Jeronimo C, Harden S, Engles J, Danenberg P V, Yang S, Sidransky D. (2002) Quantitative Adenomatous Polyposis Coli Promoter Methylation Analysis in Tumor Tissue, Serum, and Plasma DNA of Patients with Lung Cancer. Cancer Res., 62, 371-375.
7. Frommer, M., Mcdonald, L. E., Millar, D. S., Collis, C. M., Watt, F., Grigg, G. W., Molloy, P. L. and Paul, C. L. (1992) A genomic sequencing protocol that yields a positive display of 5-methylcytosine residues in individual DNA strands. Proc. Natl. Acad. Sci. USA, 89, 1827-1831.
8. Gonzalgo, M. L. and Jones, P. A. (1997) Rapid quantitation of methylation differences at specific sites using methylation-sensitive single nucleotide primer extension (Ms-SNuPE). Nucleic Acids Res., 25, 2529-2531.
9. Adorján, P., Distler, J., Lipscher, E., Model, F., Müller, J., Pelet, C., Braun, A., Florl, A. R., Gütig, D., Grabs, G., Howe, A., Kursar, M., Lesche, R., Leu, E., Lewin, A., Maier, S., Müller, V., Otto, T., Scholz, C., Schulz. W. A., Seifert, H., Schwope, I., Ziebarth, H., Berlin, K., Piepenbrock, C. and Olek, A. (2002) Tumour class prediction and discovery by microarray-based DNA methylation analysis, Nucleic Acids Res., 30, e21.
10. Eads, C. A., Danenberg, K. D., Kawakami, K., Saltz, L. B., Blake, C., Shibata, D., Danenberg, P. V., Laird, P. W. (2000) MethyLight: a high-throughput assay to measure DNA methylation. Nucleic Acids Res., 28, e32.
11. Uhlmann, K, Marczinek, K., Hampe, J., Thiel, G., Numberg, P. (1999) Changes in methylation patterns identified by two-dimensional DNA fingerprinting. Electrophoresis, 20, 1748-1755.
12. Uhlmann, K, Rohde, K., Zeller, C., Szymas, J., Vogel, S., Marczinek, K., Thiel, G., Nümberg, P., Laird, P. W. (submitted) Distinct Methylation Profiles of Glioma Subtypes. Int. J. Cancer
13. Kleihues, P., Burger, P. C., Scheithauer, B. W. (1993) The new WHO classification of brain tumours. Brain Pathol., 3, 255-268.
14. Marczinek, K., Hampe, J., Uhlmann, K., Thiel, G., Barth, I., Mrowka, R., Vogel, S., Numberg, P. (1998) Genomic difference analysis by two-dimensional DNA fingerprinting reveals typical changes in human low-grade gliomas. Glia, 1998, 23, 130-138.
15. Olek, A., Oswald, J., Walter, J. (1996) A modified and improved method for bisulphite based cytosine methylation analysis. Nucleic Acids Res., 24, 5064-5066.
16. Mátyás, G., Giunta, C., Steinmann, B., Hossle, J. P. and Hellwig, R. (2002) Quantification of Single Nucleotide Polymorphisms: A Novel Method That Combines Primer Extension Assay and Capillary Electrophoresis. Hum. Mut 19, 58-68.
17. Makridakis, J. M. and Reichardt, J. K. V. (2001) Multiplex Automated Primer Extension Analysis: Simultaneous Genotyping of Several Polymorphisms. BioTechniques, 31, 1374-1380.

1. A method for the detection of the methylation status of a nucleotide at a predetermined position in a nucleic molecule comprising the steps of
(a) treating a sample comprising said nucleic acid molecule or consisting of said nucleic acid molecule in an aqueous solution with an agent suitable for the conversion of said nucleotide if present in
(i) methylated form; or
(ii) non-methylated form
to pair with a nucleotide normally not pairing with said nucleotide prior to conversion;
(b) amplifying said nucleic acid molecule treated with said agent;
(c) real-time sequencing said amplified nucleic acid molecule; and
(d) detecting whether said nucleotide is methylated or not methylated in said predetermined position in the sample.

2. The method of claim 1 wherein said sample is derived from a tissue, a body fluid or stool.

3. The method of claim 2 wherein said tissue is a tumor tissue, a neurodegenerative tissue or a tissue affected with another neurological disorder.

4. The method of claim 1 wherein said nucleic acid molecule is a DNA molecule or an RNA molecule.

5. The method of claim 1 wherein the amplification in step (b) is effected by LCR or PCR.

6. The method of claim 5 wherein one amplification primer is detectably labeled.

7. The method of claim 6 wherein said label is biotin, avidin, streptavidin or a derivative or a magnetic bead.

8. The method of claim 1 wherein said methylated nucleotide is an adenine, guanine or a cytosine.

9. The method of claim 1 wherein said real-time sequencing comprises:
(a) hybridization of a sequencing primer to said amplified nucleic acid molecule in single-stranded form;
(b) addition of a DNA polymerase, a ATP sulfurylase, a luciferase, an apyrase, adenosine-phosphosulfate (APS) and luciferin;
(c) sequential addition of all four different dNTPs;
(d) detection of a luminescent signal wherein the intensity of the luminescent signal is correlated with the incorporation of a specific nucleotide at a specific position in the nucleic acid molecule and wherein the intensity of said signal is indicative of the methylation status of said nucleotide in said predetermined position.

10. The method of claim 1 further comprising quantifying the methylated nucleotides.

11. The method of claim 1 wherein said agent suitable for the conversion of said nucleotide to pair with a nucleotide normally not pairing with said nucleotide is a bisulfite, preferably sodium bifulfite.

12. A method for the diagnosis of a pathological condition or the predisposition for a pathological condition comprising detection of the methylation status of a nucleotide at a predetermined position in a nucleic acid molecule comprising the steps of
(a) treating a sample comprising said nucleic acid molecule or consisting of said nucleic acid molecule in an aqueous solution with an agent suitable for the conversion of said nucleotide if present in
(i) methylated form; or
(ii) non-methylated form
to pair with a nucleotide normally not pairing with said nucleotide prior to conversion;
(b) amplifying said nucleic acid molecule treated with said agent;
(c) real-time sequencing said amplified nucleic acid molecule; and
(d) detecting whether said nucleotide is methylated or not methylated in said predetermined position in the sample wherein a methylated or a not methylated nucleotide is indicative of a pathological condition or the predisposed for said pathological condition.

13. The method of claim 12 wherein said pathological condition is cancer, a neurodegenerative disease or another neurological disorder.

14. The method of claim 13 wherein said cancer is a primary tumor, a metastasis or a residual tumor.

15. The method of claim 14 wherein said primary tumor is a glioma.

16. The method of claim 15 wherein said glioma is an astrocytoma, oligodendroglioma, an oligoastrocytoma, a glioblastoma, a pilocytic astrocytoma.

17. The method of claim 13 wherein said neurodegenerative disease is Alzheimer's disease, Parkinson disease, Huntington disease, or Rett-Syndrome.

18. The method of claim 13 wherein said neurological disorder is Prader-Willi-Syndrome, Angelman-Syndrome, Fragile-X-Syndrome, or ATR-X-Syndrome.

19. The method of claim 12 wherein said nucleic acid molecule is a DNA molecule or an RNA molecule.

20. The method of claim 12 wherein the amplification in step (b) is effected by LCR or PCR.

21. The method of claim 20 wherein one amplification primer is detectably labeled.

22. The method of claim 21 wherein said label is biotin, avidin, streptavidin or a derivative or a magnetic bead.

23. The method of claim 12 wherein said methylated nucleotide is an adenine, guanine or a cytosine.

24. The method of claim 12 wherein said real-time sequencing comprises:
(a) hybridization of a sequencing primer to said amplified nucleic acid molecule in single-stranded form;
(b) addition of a DNA plymerase, a ATP sulfurylase a luciferase, an Apyrase, adenosine-phosphosulfate (APS) and luciferin;
(c) sequential addition of all four different dNTPs'
(d) detection of a luminescent signal wherein the intensity of the luminescent signal is correlated with the incorporation of a specific nucleotide at a specific position in the nucleic acid molecule and wherein the intensity of said signal is indicative of the methylation status of said nucleotide in said predetermined position.

25. The method of claim 12 further comprising quantifying the methylated nucleotides.

26. The method of claim 12 wherein said agent suitable for the conversion of said nucleotide to pair with a nucleotide normally not pairing with said nucleotide is a bisulfite, preferably sodium bisulfite.

27. The method of claim 1 wherein said method is a high-throughput method.


SEQUENCE LISTING (4 sequences)

SEQ ID NO: 1 - DNA, 28 bp, artificial
  Synthetic
tgagttggaataagttagggtagatgtg

SEQ ID NO: 2 - DNA, 32 bp, artificial
  Synthetic
caactctctatatccctttctaacataaatca

SEQ ID NO: 3 - DNA, 20 bp, artificial
  Synthetic
ttaggggggtgaatattggg

SEQ ID NO: 4 - DNA, 15 bp, artificial
  Synthetic
ggggtgaatattggg